(19) United States
(12) Patent Application Publication
(10) Pub. No.: US 2011/0311611 A1
(43) Pub. Date: Dec. 22, 2011

(54) WATER DISSOLVABLE ADHERENT ARTICLE FOR TOPICAL APPLICATION

(76) Inventors: Charlene Cheuk Ling Ng, Scotch Plains, NJ (US); John T. Ulman, Woodbridge, NJ (US)

(21) Appl. No.: 12/820,434

(22) Filed: Jun. 22, 2010

(51) Int. Cl. A61K 9/70; A61M 35/00; A61P 29/00; A61P 23/02; A61P 31/04; A61P 31/12

(52) U.S. Cl. 424/448; 604/307

(57) ABSTRACT

An adherent article with a first substrate, soluble or dispersible in aqueous solutions, with a first and second side, and an aqueous-insoluble second substrate disposed on at least a portion of the second side of the first substrate, optionally, with an adhesive applied to at least a portion of the first side of the first substrate, and a release liner positioned over a portion of the first substrate or adhesive to cover and protect at least a portion of the adhesive.

### FIELD OF THE INVENTION

This invention relates to a water-dissolvable adherent article for topical applications.

### BACKGROUND OF THE INVENTION

Adhesive patches or bandages are widely used for topical applications such as to cover and protect wounds, cuts, blisters, blemishes, and the like, or for topical delivery of bioactives or drugs on various parts of the human body. A variety of adhesive patch structures and designs are commercially available to attend to different patient needs, based on the location and application. Such patches typically include an adhesive material that is applied to a substrate material. To utilize the patch, a consumer first positions a portion of the substrate material over the site of use. Then, the consumer places the patch on the site with the adhesive material in contact with the surrounding areas of skin. The adherence of the substrate material to the skin allows the patch to be tightly fitted over site, allowing the patch to protect the wound from infection, or deliver bioactives or drugs to the desired location.

One issue associated with conventional adherent patches relates to the removal of them from the skin. In particular, a consumer typically forcibly tears the article away from the skin to break the adhesive bond of the substrate to the skin. This tearing often imparts pain to the user. The pain may be due to sensitive or fragile skin, where any pulling of the sticky patch can also induce skin injury. In addition, in sections of the skin with significant hair, forceful removal of an adherent topical patch may depilate the area.

Known in the art are adherent articles that can be more easily removed than conventional adherent articles. These include water soluble patches, tapes and bandages (see U.S. Pat. No. 6,576,575, for example).

The water-soluble patches known in the art lack the ability for the consumer to control the point in time when they wish to remove the patch, as the substrate is exposed to moist environmental conditions (rain, etc.) and may additionally be exposed to water in sinks, showers, sweating, etc. while in use.

There is a need for a patch that can be easily removed from the site of use, where the consumer is able to control the point in time when they wish to remove the patch. In response to the discussed difficulties and problems encountered in the prior art, a new adherent article has been now discovered, as described herein below.

### SUMMARY OF THE INVENTION

The adherent article of the present invention has a first substrate with a first side for contacting skin and second side opposite the first side, where the first substrate is soluble in aqueous solutions, and a second substrate disposed on at least a portion of the second side of the first substrate, the second substrate being substantially insoluble in aqueous solutions. An adhesive can be applied to at least a portion of the first side of the first substrate, with the adhesive being capable of adhering the first substrate to human skin.

### BRIEF DESCRIPTION OF THE DRAWINGS

FIG. 1 is a side view of a first embodiment of an adherent article of the present invention.

FIG. 2 is a top view of a second embodiment of an adherent article of the present invention.

FIG. 3 is a cross-sectional view along the 3- - - 3 plane of the embodiment of FIG. 2.

FIG. 4 is a side view of a third embodiment of an adherent article of the present invention.

FIG. 5 is a top view of a fourth embodiment of an adherent article of the present invention.

FIG. 6 is a cross-sectional view along the 6- - - 6 plane of the embodiment of FIG. 5.

### DETAILED DESCRIPTION OF THE INVENTION

As used herein, the phrase “aqueous-solution-dissolvable” or “aqueous-solution-soluble” refers to materials that are soluble or dispersible in aqueous solutions below 60° C. The phrase “aqueous-solution-non-dissolvable” or “aqueous-solution-insoluble” refers to materials that are insoluble or non-dispersible in aqueous solutions below 60° C. Aqueous solutions can include any liquid or solution, such as water or solutions thereof.

The present invention is directed to an adherent article that includes a substrate applied with an adhesive, often a pressure-sensitive adhesive, which can allow the substrate to be retained on human skin. The adherent article may be in the form of a tape or wound covering device, such as a bandage or patch. An adherent article of the present invention can at least partially dissolve and/or disperse in aqueous solution at a time chosen by the user. At the chosen time, the user removes the article from their skin without causing substantial pain or irritation. The adherent article can also retain enhanced strength characteristics and dimensional integrity during its use. For example, in one embodiment, a portion of a first substrate is made from an aqueous-solution-soluble natural or synthetic polymer. A second substrate, made from an aqueous-solution-insoluble natural or synthetic polymer, is applied to one side of the first substrate. An adhesive, such as an acrylate polymer, is applied to the other skin-contacting side of the first substrate to form a skin bandage. Upon removal of the second substrate, the first substrate dissolves and/or disperses when contacted with substantial amounts of water or aqueous solution. As used herein, “substantial amounts of water” refers to levels of water than may be used during bathing, showering and the like. It is not intended to include levels of moisture attained, for example, through perspiration of a person, or high relative humidity. The adherent article can also include a pad to aid in absorbing or delivering medicines and/or absorbing blood or fluids from a cut or wound, and/or a release liner that is positioned over a portion of the second side of first substrate to cover and protect at least a portion of the first substrate or the adhesive.

To use the adherent article, the user peels away the liner from the substrate and applies the adherent article to the skin. At a time chosen by the user, the user removes the insoluble second substrate from the adherent article. Upon removal of the second substrate, the first substrate dissolves and/or disperses when contacted with substantial amounts of water, allowing removal of the adherent article from their skin without causing substantial pain, irritation, or depilation.

The adherent article may be in the form of a patch for topical application, with the patch having a first substrate with a first and second side, the first substrate being an aqueous-solution-soluble film, a second substrate disposed on at least a portion of the first side of the first substrate, the second substrate being an insoluble, or substantially insoluble, film, and an adhesive applied to at least a portion of the second side of the first substrate, the adhesive being capable of adhering the first substrate to human skin.

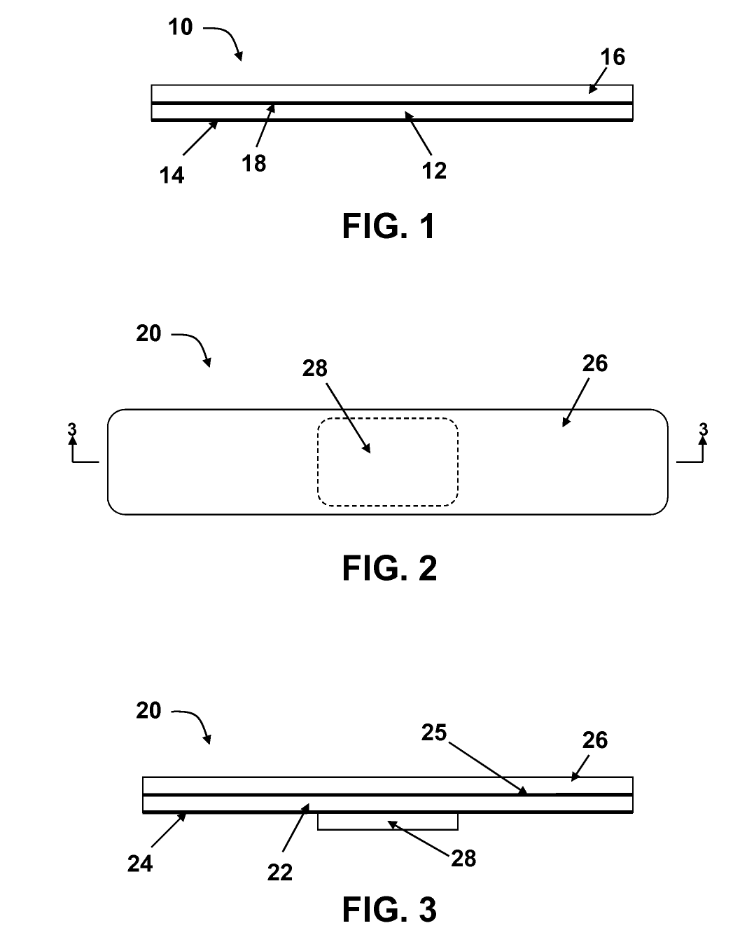

In accordance with the present invention, an adherent article of the present invention includes a first substrate that is soluble in aqueous solutions, i.e. aqueous-solution-soluble, and a second substrate that is insoluble in aqueous solutions, i.e. aqueous-solution-insoluble. Referring to FIG. 1, adherent article 10, in the form of a patch, contains a first substrate 12 that is soluble in aqueous solutions. A first adhesive 14 is disposed on a first side of first substrate 12. A second adhesive 18 is disposed on a second side of first substrate 12. A second substrate 16 that is insoluble in aqueous solutions is disposed on second adhesive 18 such that second side of first substrate 12 is essentially covered by substrate 16. Though FIG. 1 shows second side of first substrate 12 essentially covered by second substrate 16, it is to be understood that second side of first substrate 12, may be partially, essentially, or fully covered by second substrate 16.

FIG. 1 also shows first side of first substrate 12 fully covered by first adhesive 14, and second side of first substrate 12 fully covered by second adhesive 18. It is to be understood that first and/or second side of substrate 12 may be fully, essentially, or partially covered by first adhesive 14 and/or second adhesive 18. It is also to be understood that adherent article 10 may have no adhesive component, i.e. article 10 does not have first adhesive 14 or second adhesive 18.

When substantially dry, substrates 12 and 16 provide increased dimensional stability and strength to adherent article 10 in dry or wet environments. When second substrate 16 is removed from first substrate 12, substrate 12 dissolves when contacted with substantial amounts of water or aqueous solutions.

In general, any material that is dissolvable in aqueous solution can generally be used to form first substrate 12. For example, in some embodiments, substrate 12 can comprise cold-aqueous-solution-soluble, or hot-aqueous-solution-soluble materials in the form of films, wovens, non-wovens, or foams. As used herein, “cold-aqueous-solution-soluble”generally refers to materials that can substantially dissolve or disperse in an aqueous solution at temperatures of less than about 40° C. In particular, the cold-water-soluble materials for use as substrates in embodiments of the present invention are normally capable of dissolving at temperatures between about 15° C. to about 40° C. Thus, for example, one-cold-water-soluble substrate might dissolve at temperatures of 35° C. or higher. Another cold-water-soluble substrate, for example, might dissolve at temperatures of 15° C. or higher.

In addition to materials soluble in cold aqueous solution, first substrate 12 can also comprise synthetic films soluble in hot aqueous solution. As used herein, “hot-aqueous-solution-soluble films” generally refers to films that substantially dissolve or disperse in an aqueous solution only at temperatures greater than about 40° C. For instance, in some embodiments, the hot-aqueous-solution-soluble films only substantially dissolve in an aqueous solution at temperatures greater than about 50° C.

Any natural or synthetic material that can be substantially dissolved in an aqueous solution can generally be used in the present invention as the aqueous-solution-soluble first substrate 12. Commercially available films that are suitable for use as aqueous-solution-soluble films, wovens, non-wovens, or foams in the present invention are polyvinyl alcohol (PVOH), water-dispersible sulfopolyester, gelled alginate, polyoxyethylene (PEO), methylcellulose (MC), hydroxypropylmethylcellulose (HPMC), pullulan, pectin and starches.

In one embodiment, the substrate, in the form of a film soluble in aqueous solution, is comprised of polyvinyl alcohol. In particular, the aqueous-solution-soluble polyvinyl alcohol films can, in some instances, substantially dissolve in aqueous solution at a temperature of at least about 20° C. to about 50° C.

Regardless of the material utilized, first substrate 12 generally contains polymers, whether cold-aqueous-solution-soluble or hot-aqueous-solution-soluble, in an amount of about 50% or more by weight of the substrate. In particular, first substrate 12 can contain about 70% or more by weight of aqueous-solution-soluble polymers, and more particularly, about 80% or more by weight of aqueous-solution-soluble polymers. For example, in one embodiment, the first substrate 12 contains about 100% by weight of aqueous-solution-soluble polymers.

In general, any material that is aqueous-solution-insoluble can generally be used in second substrate 16. For example, in some embodiments, substrate 16 can comprise aqueous-solution-insoluble, or substantially insoluble, films. The term “substantially insoluble”, as used herein, mean a material that will not dissolve when immersed in water at 25° C. for less than 24 hours, but will at least partially dissolve after 24 hours.

The second substrate 16 could be comprised of hydrophobic or hydrophilic materials. Materials include, but are not limited to, nylon, polypropylene (PP), polyethylene (PE), polyurethane (PU), poly(tetrafluoroethylene), polyvinylidene fluoride, polyvinyl chloride (PVC) and vinyl/acrylic co-polymers. The second substrate 16 could be solid, or have pores of various numbers or sizes. Porous films would be expected to speed the dissolution of first substrate 12.

In one embodiment, films used as second substrate 16 are comprised of polyethylene (PE). Commercially available polyethylene films that are suitable for use as second substrate 16 in the present invention include 3M Protective UV Tape (2AU26B, 3M, Minneapolis, Minn.).

Regardless of the material utilized, second substrate 16 generally contains polymers insoluble in aqueous solutions, in an amount of about 50% or more by weight of the substrate. In particular, substrate 16 can contain about 70% or more by weight of aqueous-solution-insoluble polymers, and more particularly, about 80% or more by weight of aqueous-solution-insoluble polymers. For example, in one embodiment, substrate 16 contains about 100% by weight of polymers insoluble in aqueous solutions below 60° C.

In some embodiments, second substrate 16 could be a textured or smooth film. Second substrate 16 could be clear or have a color to identify it when it is attached to the first substrate 12. It could change color when exposed to sunlight, heat or cold. In some embodiments, graphic designs or pictures attached or printed on the side of second substrate 16 not in contact with first substrate 12. In other embodiments, second substrate 16 could be clear, and graphic designs or pictures are attached or printed on the second side of first substrate 12.

In some embodiments, second substrate 16 could be a film formed by spraying a material onto first substrate 12. A material for use in this embodiment is silicone.

In some embodiments, second substrate 16 could have an aided lifting zone so that it is easy to lift and remove, or peel, second substrate 16 from first substrate 12. The aided lifting zone may be in the form of a lip, grip, or handle that can be attached to an edge of the distal edge of adherent article 10 to ease the peeling process. This lip would develop a double ply or two lays with a width and length suitable for a consumer to easily grip it in any condition, such as the shower, so it can be removed to allow the aqueous-solution-dissolvable first substrate 12 to be washed off. The lip edges could be applied to either edge of a bandage or on the edge of any shaped patch or bandage.

Second substrate 16 is disposed on a first side of first substrate 12 such that first side of substrate 12 is partially or fully covered by substrate 16. First substrate 12 and second substrate 16 are bonded either physically or chemically.

In some embodiments, first substrate 12 and second substrate 16 are bonded using known adhesives. In other embodiments, one or both substrates are cling films, which typically are films made of polymers such as polyethylene (PE) and polyvinyl chloride (PVC) which are so thin so as to be characterized by their ability to stick (or cling) to any object with a smooth surface without adhesive. Here, the adhesion forces are so called Van-der-Waals forces, due to polar molecules which induce weak electrostatic forces between two surfaces. Cling film can be removed without leaving any residue and is reusable.

In still other embodiments, first substrate 12 and second substrate 16 are bonded by heat, pressure, or by use of solvents or co-solvents. In these cases, no adhesive is needed.

In use, adherent article 10 is placed on the skin of the user at the site of use with first substrate 12 in contact with the user's skin. If first substrate 12 is in the form of a cling film, substrate 12 will adhere to the user's skin.

In some embodiments, second substrate 16, as well as first substrate 12, are in contact with the user's skin. If second substrate 16 is in the form of a cling film, second substrate 16 will adhere to the user's skin.

In some embodiments, adhesion of first substrate 12 and/or second substrate 16 to a user's skin, or to each other, is facilitated by use of an adhesive applied to either or both substrates. Although not required, the adhesive may be a pressure-sensitive adhesive. A pressure-sensitive adhesive is an adhesive that is normally tacky at room temperature (e.g., about 20° C.) and that can form a bond to a surface by the application of small amounts of pressure.

In general, any of a variety of pressure-sensitive adhesives can be utilized in the present invention as first adhesive 14 and as second adhesive 18. In particular, pressure-sensitive adhesives that are biocompatible with human skin are typically utilized. Moreover, an adhesive of the present invention is also either substantially water soluble or substantially water insoluble, but dispersible in an aqueous environment. For example, one suitable adhesive disclosed includes about 10-75% by weight of a polyalkyloxazoline polymer, 10-75% by weight of a functional diluent comprising a hydroxy compound or a carboxylic acid compound, and 5-50% by weight of a tackifier.

The water-dispersible polymeric component can include, for example, surfactants such as poly(ethylene oxide) alkylphenyl ethers, such as those sold under the trade names IGEPAL.CO and IGEPAL.CA (available from Rhodia, La Defense, France); poly(ethylene oxide) lauryl, cetyl, and oleyl ethers such as those sold under the trade name BRIJ (available from ICI Americas, Inc., Wilmington, Del.); poly(ethylene oxide) laurate; poly(ethylene oxide) oleate; sorbitan oleate; ethylene oxide/propylene oxide block copolymers such as those sold under the trade names PLURONIC and TETRONIC (available from BASF Corporation, Florham Park, N.J.); and organic phosphate esters, such as those sold under the trade name GAFAC PE-510 (available from International Specialty Products, Wayne, N.J.). Examples of other components include, but are not limited to, poly(acrylic acid); poly(vinyl alcohol); poly(N-vinyl pyrrolidone); poly(acrylamide); poly(alkoxyalkyl (meth)acrylates), such as 2-ethoxy ethyl acrylate, 2-ethoxy ethyl methacrylate, 2-(2-ethoxyethoxy)ethyl acrylate, and 2-methoxy ethyl acrylate (available from Sartomer Company, Inc., Exton, Pa.); poly(vinyl methyl ether); poly(vinyl methyl ether: maleic anhydride), sold under the trade name GANTREZ (available from International Specialty Products, Wayne, N.J.); poly(ether polyols), such as polypropylene glycol) and the like, such as those sold under the trade name SANNIX (available from Sanyo Chemical Industries, Kyoto, Japan); copolymers thereof, and the like. Copolymers of these and alkyl (meth)acrylate esters or vinyl esters are also suitable. Gums such as those derived from okra and guar may also be used.

Still another suitable pressure-sensitive adhesive includes about 10% to about 80%, by weight, of an alkali soluble polymer; about 0 to about 30%, by weight, of a poly(vinyl methyl ether); about 30% to about 70%, by weight, of a tackifying resin; and about 5% to about 30%, by weight, of a suitable plasticizer. Still other examples of suitable adhesives include HX 9236-01 or HX 9237-01 hot melt adhesives, which are obtainable from ATO Findley, Inc. (Wauwatosa, Wis.).

In general, as stated above, first adhesive 14 and second adhesive 18 can be applied to one or both sides of the substrate 12. In some embodiments, first adhesive 14 is applied only to the first side of first substrate 12 opposite to the side on which second substrate 16 or second adhesive 18, is disposed. The first side of first substrate 12 typically is designed to adhere to human skin. First adhesive 14 and second adhesive 18 can be applied to first substrate 12 through any known method of application, such as print, film, spray, saturate, coat, droplet throw, and foam applications. In one embodiment, for example, first adhesive 14 is sprayed onto first substrate 12 using a melt spray applicator. In another embodiment, first adhesive 14 is applied as a film layer to first substrate 12.

In some embodiments, second adhesive 18 is applied to second substrate 16, and the second substrate 16/second adhesive 18 combination is disposed on second side of first substrate 12. Second adhesive 18 can generally be applied to second substrate 16 through any known method of application, such as direct, print, film, spray, transfer, saturant, coat, droplet throw, and foam applications.

In addition, depending on the desired adherent characteristics of the resulting article, first adhesive 14 and second adhesive 18 can be applied in any of a variety of amounts to first substrate 12 or second substrate 16. The amount of adhesive coated is typically 1 to 3 mil in thickness. For example, in some embodiments, first adhesive 14 can be applied in an amount between about 3 grams per square meter to about 50 grams per square meter, and particularly between about 5 grams per square meter to about 25 grams per square meter. Further, first adhesive 14 and/or second adhesive 18 can also be applied either uniformly or non-uniformly to first substrate 12 or second substrate 16.

In use, adherent article 10 is placed on the skin of the user at the site of use (wound, cut, blister, blemishes, etc.) with first substrate 12 in contact with the user's skin. As a result of the present invention, adherent article 10 can generally be removed from a user's skin by using substantial amounts of an aqueous solution, such as water from a shower, bath, faucet, etc. At a time chosen by the user, the user removes the second substrate 16 from the adherent article 10. Upon removal of the second substrate, the first substrate dissolves and/or disperses when contacted with substantial amounts of aqueous solution or water, allowing removal of the adherent article from their skin without causing substantial pain, irritation, or depilation. For example, when a sufficient amount of water contacts first substrate 12, the aqueous solution soluble first substrate 12 substantially dissolves. Moreover, the same solution can also cause first adhesive 14 to disperse and lose its strength and adherent properties, thereby allowing first substrate 12 to be even more easily removed.

The adherent article of the present invention can also contain a variety of other components. In an alternative embodiment, the adherent article can contain a pad to aid in absorbing or delivering medicines and/or absorbing blood or fluids from a cut or wound. In a second embodiment, shown in FIGS. 2 and 3, adherent article 20 comprises a first substrate 22 that is soluble in aqueous solutions. A first adhesive 24 is disposed on a first side of first substrate 22. A second adhesive 25 is disposed on a second side of first substrate 22. A second substrate 26, that is insoluble in aqueous solutions, is disposed on second adhesive 25 such that second side of first substrate 22 is essentially covered by second substrate 26. Though FIGS. 2 and 3 show second side of first substrate 22 essentially covered by second substrate 26, it is to be understood that second side of first substrate 22, may be partially, essentially, or fully covered by second substrate 26. Pad 28 is disposed on first adhesive 24 such that first side of first substrate 22 covers pad 28. FIGS. 2 and 3 show pad 28 is fully covered by first side of first substrate 22. It is to be understood, however, that pad 28 may be partially, essentially, or fully, covered by first substrate 22.

Pad 28 can be made from any type of material commonly used in the art in forming such pads. These include gauzes of cotton, wool, silk or synthetic fiber, poultice, fluff wood pulp, non-wovens, and absorbent hydrogels, such as gelled alginate based foams. In some embodiments, pad 28 can also be dispersible in water to facilitate in disposal of the adherent article 20. For example, water-dispersible coform composites are one example of a suitable absorbent pad that can be used in the present invention. Other suitable materials that can be used for the pad 28 include lightly cross-linked tissue structures, absorbent films, and the like.

In still other embodiments, absorbent pad 28 can be made cellulose fiber or non-biodegradable or biodegradable foam. Also, absorbent pad 28 may be wrapped or laminated with a cover of plastic or treated cellulose to prevent bonding to the wound tissue.

FIG. 3 shows first adhesive 24 disposed on first side of first substrate 22. Second adhesive 25 is disposed on the second side of first substrate 22. First adhesive 24 is designed to adhere adherent article 20 to human skin. Second adhesive 25 is used to adhere first substrate 22 to second substrate 26. In FIG. 3, first adhesive 24 is applied to the first side of first substrate 22, not covering pad 28. In some embodiments, first adhesive 24 may partially, substantially, or fully cover pad 28.

When substantially dry, first substrate 22 and second substrate 26 provide increased dimensional stability and strength to adherent article 20 in dry or wet environments. When second substrate 26 is removed from first substrate 22, first substrate 22 dissolves when contacted with substantial amounts of water or aqueous solutions, and adherent article 20 disintegrates.

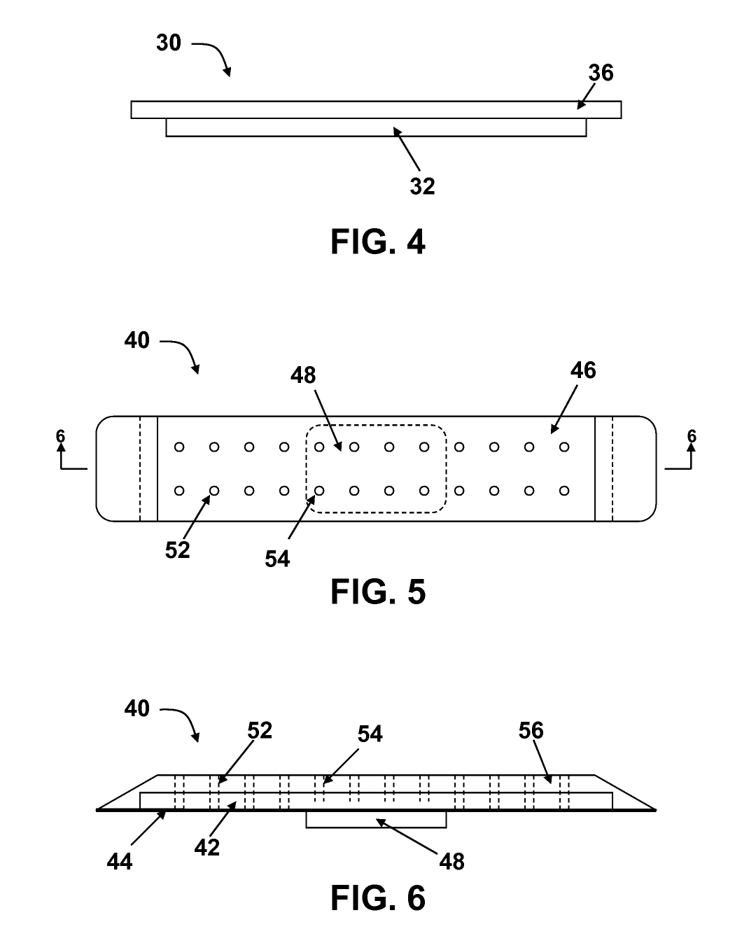

As mentioned, the adherent article of the present invention can also contain no adhesive. In a third embodiment, shown in FIG. 4, adherent article 30 comprises a first substrate 32 that is soluble in aqueous solutions. A second substrate 36, that is insoluble in aqueous solutions, is disposed on a first side of first substrate 32 such that first side of substrate 32 is fully covered by substrate 36. Though FIG. 4 shows second substrate 36 fully covering first side of substrate 32, it is to be understood that first side of substrate 32, may be partially, essentially, or fully, covered by substrate 36.

In this embodiment, first substrate 32 and second substrate 36 are not bonded by using known adhesives. Here, first substrate 32 and second substrate 36 are bonded by heat, pressure, or by use of solvents or co-solvents. In other embodiments, one or both substrates are cling films, as discussed above, characterized by their ability to stick to smooth surfaces without adhesive.

In use, the embodiment of FIG. 4 is placed on the user's skin with first substrate 12, or both first substrate 12 and second substrate 16 are in contact with the user's skin.

In a fourth alternative embodiment, the adherent article can contain both a pad and pores through one or both substrates to aid in the ability of the adherent article to breathe. This may prevent maceration, the process in which the skin is softened and broken down by extended exposure to wetness or moisture, of the skin under the patch.

As shown in FIGS. 5 and 6, adherent article 40 comprises a first substrate 42 that is soluble in aqueous solutions. Adhesive 44 is applied to the first side of first substrate 42. A second substrate 46, that is insoluble in aqueous solutions, is disposed on second side of first substrate 42 such that second side of substrate 42 is fully covered by substrate 46. Though FIG. 5 shows second substrate 46 fully covering second side of substrate 42, it is to be understood that second side of substrate 42, may be partially, fully, or essentially fully, covered by substrate 46. Pad 48 is disposed on adhesive 44 such that first side of substrate 42 covers pad 48. FIG. 5 shows pad 48 is fully covered by second side of substrate 42. It is to be understood, however, that pad 48 may be partially, fully, or essentially fully, covered by substrate 42.

In FIG. 6, first adhesive 44 is applied to the first side of first substrate 42, not covering pad 48. In some embodiments, first adhesive 44 may partially, substantially, or fully cover pad 48.

FIGS. 5 and 6 also show pores such as 52 and 54 which fully penetrate second substrate 46 and first substrate 43. Pores 52 and 54 may aid in the ability of the adherent article to breathe, possibly preventing maceration. Though shown as having circular cross-sections, pores 52 and 54 may be of any cross-section, and may be placed in an ordered or random pattern on the first side of second substrate 46. In addition the pores may be sized to allow some moisture to penetrate second substrate 46, or sized only large enough to allow for the passage of vapor through adherent article 40. Pores may also be located only in the region adherent article 40 comprising pad 48.

When substantially dry, substrates 42 and 46 provide increased dimensional stability and strength to adherent article 40 in dry or wet environments. When second substrate 46 is removed from first substrate 42, substrate 42 dissolves when contacted with substantial amounts of water or aqueous solutions, and adherent article 40 dissolves or disintegrates.

One or more active ingredients can be added to the films or the adhesive of the adherent article of the present invention. Alternatively, active ingredients can be attached to the surface of the adhesive by printing or attaching an additional layer of hydrogels, foam, or pads to the adherent article. These actives could cover the complete surface of the adherent article, or a selective area on the adherent article. These actives can be used to speed up the cure of a cold sore or shingles. A variety of topical healthcare conditions, such as wound care, blister, acne, corn, or foot care can be treated by actives disposed on the adherent articles of the invention. Typical actives include: antibacterial agents, anti-viral agents, anti-fungal agents, healing agents, pain relieving agents, anesthetics, non-steroidal anti-inflammatory drugs, and anti-inflammatory drugs.

Though not shown, the adherent articles of the present invention can also include a release liner that is positioned over a portion of the first substrate to cover the pressure-sensitive adhesive. By covering the pressure-adhesive, the release liner can ensure that the adherent properties of the adhesive remain in tact until utilized. To use the adherent article of the present invention, a user can simply peel away the liner and adhere the substrate to the skin at by contacting the adhesive portions of the substrate to the skin. In general, the release liner can be made from any material commonly used in the art as a liner for an adhesive bandage. Moreover, although not required, the release liner may be generally dispersible in water to further enhance waste disposal.

To use the adherent article, a user peels away the liner from the adhesive-coated substrate and applies the adherent article to the skin. At a time chosen by the user, the user removes the insoluble second substrate from the adherent article, and the first substrate dissolves and/or disperses when contacted with substantial amounts of water, allowing removal of the adherent article from their skin without causing substantial pain, irritation, or depilation.

The present invention may be better understood with reference to the following examples.

### Example 1

An adherent article of the embodiment of FIGS. 2 and 3 of the present invention was assembled as follows.

Second substrate 26, pre-coated with second adhesive 25, was 3M Protective UV Tape 2AU26B, with dimensions of 2 cm by 6 cm by 1-mil thick. Second substrate 26 was polyethylene, and second adhesive 25 was acrylic adhesive. First substrate 22 was a water-dispersible sulfopolyester dry film described in U.S. Patent Application Nos. US20070259029A1 and US20070258935A1, the content each of which is incorporated by reference herein in its entirety herein, with dimensions of 2 cm by 6 cm by 2-mil thick. Second adhesive 25 adhered first substrate 22 to second substrate 26. First adhesive 24, disposed on the first side of first substrate 22, was a dissolvable pressure-sensitive adhesive described in U.S. Pat. No. 6,525,129B1, the content of which is incorporated by reference herein in its entirety, and EPO Application No. EP1242555B1, a 1.5-mil thick transfer coating. Pad 28 was an absorbant gelled alginate based foam described in U.S. Patent Application No. US20070066694A1, the content of which is incorporated by reference herein in its entirety, with dimensions of 1.2 cm by 1.5 cm by 15-mil thick.

First adhesive 24 was transfer coated onto the first side of first substrate 22. Second substrate 26 with second adhesive 25 was assembled onto second side of first substrate 22. Pad 28 was presses onto first adhesive 24 to attach it to patch 20.

This construction can be used as a dissolvable wound covering or bandage. When placed on skin, the construct will stick to the skin and provide consumer perceivable benefits such as cushioning, seal out dirt and germs, fluid absorption, and hurt-free removal. Second substrate 26 acts as a protective cover on first substrate 22. With the protective cover on, the bandage is resistance to water. When the protective cover is peeled off, the entire construction will dissolve when washed.

### Example 2

An adherent article of the embodiment of FIG. 1 of the present invention was assembled as follows.

Second substrate 16, pre-coated with second adhesive 18, was 3M Protective UV Tape 2AU26B, with dimensions of 20 mm diameter by 1-mil thick. Second substrate 16 was polyethylene, and second adhesive 18 was acrylic adhesive. First substrate 12 was a water-dispersible Polyvinyl alcohol film containing neomycin (3.5 mg), bacitracin (400 U), and polymyxin B (5000 U) per g of film material. The dimensions of first substrate 12 were 20 mm diameter by 1.5-mil thick. Second adhesive 18 adhered first substrate 12 to second substrate 16. First adhesive 14, disposed on the first side of first substrate 12, was a dissolvable pressure-sensitive adhesive described in U.S. Pat. No. 6,525,129B1, and EPO Application No. EP1242555B1, a 1.5-mil thick direct coating.

First substrate 12 was made by a conventional film making method in which film forming and active ingredients were dissolved or dispersed in a solvent. The film liquid was then cast onto a liner and the solvent was evaporated to form the water-dispersible first substrate 12. First adhesive 14 was directly coated on first side of first substrate 12. Second substrate 16 with second adhesive 18 was assembled onto second side of first substrate 12.

This construction can be used as a dissolvable antibiotic film delivery system. Adherent article 10 of this embodiment can be used to deliver an active to the surface of the user's skin, or to treat or prevent wound infection. When placed on skin, the construct will stick to the skin and provide other consumer perceivable benefits such as hurt-free removal. Second substrate 16 acts as a protective cover on first substrate 12. With the protective cover on, adherent article 10 is resistance to water. When the protective cover is peeled off, the entire construction will dissolve when washed.

### Example 3

A second adherent article of the embodiment of FIG. 1 of the present invention was assembled as follows.

Second substrate 16, pre-coated with second adhesive 18, was 3M Protective UV Tape 2AU26B, with dimensions of 5 cm by 10 cm by 1-mil thick. Second substrate 16 was polyethylene, and second adhesive 18 was acrylic adhesive. First substrate 12 was a water-dispersible polyvinyl alcohol film, with dimensions of 5 cm by 10 cm by 1.5-mil thick. Second adhesive 18 adhered first substrate 12 to second substrate 16. First adhesive 14, disposed on the first side of first substrate 12, was a dissolvable pressure-sensitive adhesive described in U.S. Pat. No. 6,525,129B1, and EPO Application No. EP1242555B1, contained 5% menthol, and was a 2.5 mil thick transfer coating.

First substrate 12 was made by a conventional film making method in which film forming ingredients were dissolved or dispersed in a solvent. The liquid was then cast onto a liner and the solvent was evaporated to form first substrate 12. First adhesive 14, containing 5% menthol was coated on first side of first substrate 12. Second substrate 16 with second adhesive 18 was then assembled onto second side first substrate 12.

This construction can be used as a hurt-free removal and hair-safe topical pain patch. One of the complaints from male consumers using topical pain patches is that upon patch removal from their shoulder or back, undesired depilation of the area also occurs. When placed on skin, patch will stick to the skin and provide consumer perceivable benefits such as a cooling sensation. Second substrate 16 acts as a protective cover on first substrate 12. With the protective cover on, adherent article 10 is resistance to water. When the protective cover is peeled off, the entire construction will dissolve when washed.

### Example 4

A third adherent article of the embodiment of FIG. 1 of the present invention was assembled as follows.

Second substrate 16, is a skin-tone, pattern-printed polyurethane adhesive dressing, with dimensions of 18 mm in diameter by 1.5-mil thick=. First substrate 12 was a water-dispersible polyvinyl alcohol film containing 2% salicylic acid, with dimensions of 14 mm diameter by 1.5-mil thick.

First substrate 12 was made by a conventional film making method in which film forming ingredients were dissolved or dispersed in a solvent. The liquid was then cast onto a liner and the solvent was evaporated to form first substrate 12. Second substrate 16 was then assembled onto second side first substrate 12.

This adherent article 10 can be used as a vanishing acne patch. The polyurethane dressing provides moisture wound healing properties and the underlying salicylic acid targets the resolution of the acne. When placed on skin, adherent article 10 will stick to the skin. Second substrate 16 acts as a protective cover on first substrate 12. With the protective cover on, adherent article 10 is resistance to water. When the protective cover is peeled off, the entire construction will dissolve when washed.

### Example 5

A fourth adherent article of the embodiment of FIG. 1 of the present invention was assembled as follows.

Second substrate 16, pre-coated with second adhesive 18, was 3M Protective UV Tape 2AU26B, with dimensions of 5 cm by 10 cm by 1-mil thick. Second substrate 16 was polyethylene, and second adhesive 18 was acrylic adhesive. First substrate 12 was a gelled alginate based foam, with dimensions of 5 cm by 10 cm by 15-mil thick. Second adhesive 18 adhered first substrate 12 to second substrate 16. First adhesive 14, disposed on the first side of first substrate 12, was a dissolvable pressure-sensitive adhesive described in U.S. Pat. No. 6,525,129B1, and EPO Application No. EP1242555B1, contained 5% menthol, and was a 2.5-mil thick transfer coating.

First substrate 12 was made by a conventional foam extrusion and dried. The liquid was then cast onto a liner and the solvent was evaporated to form first substrate 12. First adhesive 14, containing 5% menthol was transfer coated first side of first substrate 12. Second substrate 16 with second adhesive 18 was then assembled onto the second side first substrate 12.

This adherent article 10 construction can be used as a hurt-free removal and hair-safe topical pain patch. One of the complaints from male consumers using topical pain patches is that upon patch removal from their shoulder or back, undesired depilation of the area also occurs. In this case, first substrate 12 is a thick pad, which can act as a cushioning material. Other cushioning material such as a piece of dissolvable foam can also be used. When placed on skin, the patch will stick to the skin and provide consumer perceivable benefits such as a cooling sensation. Second substrate 16 acts as a protective cover on first substrate 12. With the protective cover on, the patch is resistance to water. When the protective cover is peeled off, the entire construction will dissolve when washed.

### Example 6

A series of adherent article embodiments of FIG. 1 of the present invention were assembled in which second substrate 16 comprised a variety of materials.

In all cases, first substrate 12 was a water-dispersible sulfopolyester dry film described in U.S. Patent Application No. US20070259029A1, Example 8, with dimensions of 2 cm by 6 cm by 2 mil thick. First adhesive 14, disposed on the first side of first substrate 12, was a dissolvable pressure-sensitive adhesive described in U.S. Pat. No. 6,525,129B1, and EPO Application No. EP1242555B1, a 1.5-mil thick transfer coating.

Second substrate 16, disposed on second side of first substrate 12, was selected from one of the following hydrophilic or hydrophobic materials:

Hydrophilic materials (about 1 mil in thickness):
(a) Nylon 0.2 μm pore size (Whatman 47 mm diameter membrane),
(b) Polyvinylidene fluoride (PVF) 0.45 μm pore size (Millipore Durapore HVLP membrane), and
(c) Vinyl/acrylic copolymer 0.45 μm pore size (Metricel membrane DM-450).

Hydrophobic materials (about 1 mil thickness):
(d) Polytetrafloroethylene (PTFE) 0.45 μm pore size (Millipore Fluoropore membrane FH),
(e) Polypropylene (PP) 0.45 μm pore size (Whatman PP membrane),
(f) Polypropylene (PP) 30 μm pore size (Millipore PP prefilters), or
(g) Polypropylene (PP) 80 μm pore size (Millipore PP prefilters).

First adhesive 14 was coated on one side of first substrate 12. Then, the various second substrates 16 were assembled on the other side of the dry film.

The washability of the construct was evaluated as follows. Each patch 10 was adhered onto a transparent plastic plate, with first adhesive 14 in contact with the surface of the plastic plate. The whole plate was immersed in a large beaker of stirring water at room temperature, and the duration of time it took the patch to start and complete dissolution were recorded. Two controls were also tested: an adherent article without a second substrate 16, and an adherent article with a second substrate 16 comprised of a nonporous hydrophobic polyethylene (PE) film (3M Protective UV Tape 2AU26B).

The results of the washability tests are shown on Table 1.

TABLE 1
Film washability measurement with different coverings

| | | | Time to Start | |
|---|---|---|---|---|
| 2^{nd} | | | Film | Time to Complete |
| Substrate | 2^{nd} Substrate | Pore Size | Dissolution | Film Dissolution |
| Code | Material | (micron) | (minutes) | (minutes) |
| — | No cover | — | 0.00 | 0.65 |

Hydrophilic Membranes

| | | | | |
|---|---|---|---|---|
| a | Nylon | 0.2 | 0.30 | 1.85 |
| b | PVF | 0.45 | 0.33 | 2.52 |
| c | Vinyl/acrylic | 0.45 | 0.97 | 2.80 |
| | copolymer | | | |

Hydrophobic Membranes

| | | | | |
|---|---|---|---|---|
| d | PTFE | 0.45 | 4.68 | 8.80 |
| e | PP | 0.45 | 10.73 | 15.97 |
| f | PP | 30 | 4.63 | 15.38 |
| g | PP | 80 | 0.98 | 3.15 |
| — | PE | nonporous | 199.33 | Dissolution not |
| | | | | completed |

The table shows that without a second substrate 16, the adherent article quickly disintegrated in water (less than 1 minute). On the contrary, an adherent article with a second substrate 16 comprised of a nonporous hydrophobic polyethylene (PE) film, was found to show little dissolution, practically allowing the first substrate 12 coated with first adhesive 14 to be immersed in water without dissolving until second substrate 16 is removed. The remaining data in Table 1 shows that various levels of water resistance can be achieved by varying the selection of the protective backing. For example, the selection of hydrophilic material such as nylon membrane as the protective cover was shown to delay the dissolution of the dry film. The pore size of the hydrophilic membrane has little influence on the dissolution properties of the water-dispersible film. However, the level of protection by using hydrophilic covers is not as prolong as using hydrophobic covers. For example, the change of cover material from PVF (a hydrophilic material) to PP (a hydrophobic membrane) at the same pore size (0.45 micron) delayed the start of film dissolution from less than one minute to more than ten minutes. The time to complete film dissolution was extended from 2.80 minutes to almost 16 minutes. It was also found that by increasing the pore size of the same hydrophobic membrane, water can penetrate the cover and speed up the water-dispersible film dissolution.

### Example 7

A fifth adherent article of the embodiment of FIG. 1 of the present invention was assembled as follows.

Second substrate 16, pre-coated with second adhesive 18, was 3M Protective UV Tape 2AU26B, with dimensions of 5 cm by 10 cm by 1-mil thick. Second substrate 16 was polyethylene, and second adhesive 18 was acrylic adhesive. First substrate 12 was a polyvinyl alcohol temperature responsive non-woven (from Kuraray, Tokyo, Japan), with dimensions of 5 cm by 10 cm by 5-mil thick. Second adhesive 18 adhered first substrate 12 to second substrate 16. First adhesive 14, disposed on the first side of first substrate 12, was a dissolvable pressure-sensitive adhesive described in U.S. Pat. No. 6,525,129B1, and EPO Application No. EP1242555B1, contained 5% menthol, and was a 2.5 mil thick direct coating.

First adhesive 14 was directly coated on the first side of first substrate 12. Second substrate 16 with second adhesive 18 was then assembled onto the second side first substrate 12.

In this embodiment, first substrate 12 dissolves only at water temperature above 40° C. When placed on skin, adherent article 10 will stick to the skin. Second substrate 16 acts as a protective cover on first substrate 12. With the protective cover on, adherent article 10 is resistance to water. When the protective cover is peeled off, the entire patch will dissolve when the temperature of water to which the patch is exposed to is above 40° C.

Various embodiments of the invention have been set forth above. Each embodiment is provided by way of explanation of the invention, not limitation of the invention. In fact, it will be apparent to those skilled in the art that various modifications and variations can be made in the present invention without departing from the scope or spirit of the invention.

What is claimed is:

1. An adherent article comprising:
a first substrate having a first side for contacting skin and second side opposite said first side, said first substrate being soluble in aqueous solutions; and
a second substrate disposed on at least a portion of said second side of said first substrate, said second substrate being insoluble in aqueous solutions.

2. The adherent article of claim 1, wherein said first substrate comprises a film comprising a material selected from the group consisting of polyvinyl alcohol, water-dispersible sulfopolyester, gelled alginate, polyoxyethylene, methylcellulose, and hydroxypropylmethylcellulose.

3. The adherent article of claim 1, wherein said second substrate comprises a film comprising a material selected from the group consisting of nylon, polypropylene, polyethylene, polyurethane, poly(tetrafluoroethylene), polyvinylidene fluoride, vinyl/acrylic co-polymers.

4. The adherent article of claim 1, wherein an adhesive is applied to at least a portion of said first side of said first substrate, said adhesive being capable of adhering said first substrate to human skin.

5. The adherent article of claim 4, wherein said adhesive comprises a pressure-sensitive adhesive that is generally non-soluble in said aqueous solution.

6. The adherent article of claim 4, wherein said adhesive is soluble in said aqueous solution.

7. The adherent article of claim 1, further comprising a pad disposed on said first side of said first substrate.

8. The adherent article of claim 4, further comprising a release liner, said release liner being positioned on said first side of said first substrate such that said release liner substantially covers said adhesive.

9. The adherent article of claim 1, further comprising pores which fully penetrate said second substrate.

10. The adherent article of claim 1, further comprising pores which fully penetrate said first substrate.

11. The adherent article of claim 1, further comprising an active ingredient selected from the group consisting of antibacterial agents, anti-viral agents, anti-fungal agents, healing agents, pain relieving agents, anesthetics, non-steroidal anti-inflammatory drugs and anti-inflammatory drugs.

12. A patch for topical application comprising:
a first substrate having a first and second side, said first substrate comprising an aqueous-solution-soluble film;
an adhesive applied to at least a portion of said first side of said first substrate, said adhesive being capable of adhering said first substrate to human skin; and
a second substrate disposed on at least a portion of said second side of said first substrate, said second substrate comprising an aqueous-insoluble, or substantially aqueous-insoluble, film.

13. The patch of claim 12, further comprising a release liner, said release liner being positioned on said adhesive such that said release liner substantially covers said adhesive.
